(19) United States
(12) Patent Application Publication
(10) Pub. No.: US 2026/0176693 A1
(43) Pub. Date: Jun. 25, 2026

(54) DETECTION OF NON-CANCER SOMATIC MUTATIONS

(71) Applicant: Zoetis Services LLC, Parsippany, NJ (US)

(72) Inventors: Kristina KRUGLYAK, La Jolla, CA (US); Wai Yi TSUI, La Jolla, CA (US); Ilya CHORNY, La Jolla, CA (US); Jason CHIBUK, La Jolla, CA (US); Andi FLORY, La Jolla, CA (US); Daniel GROSU, La Jolla, CA (US); Todd COHEN, La Jolla, CA (US); Allison O'KELL, La Jolla, CA (US)

(21) Appl. No.: 19/125,472

(22) PCT Filed: Oct. 31, 2023
(86) PCT No.: PCT/US2023/036465

§ 371 Date: Apr. 29, 2025

Related U.S. Application Data
(60) Provisional application No. 63/381,909, filed on Nov. 1, 2022.

(51) Int. Cl. C12Q 1/6883

(52) U.S. Cl. CPC C12Q 1/6883; C12Q 2600/156

(57) ABSTRACT

The methods, systems, and compositions provided herein allow improved methods for detecting non-cancer somatic mutations in a subject by measuring buffy coat somatic mutations in order to improve identification of samples with age related somatic mutations.

### BACKGROUND

### Field

The present disclosure relates to methods for detecting, characterizing, or managing non-cancer somatic mutations in a subject by analyzing copy number aberrations in a buffy coat DNA sample.

### Description of Related Art

Somatic mutations are a normal consequence of aging despite there being effective DNA repair machinery present in human cells. While most somatic mutations are silent, some affect genes that are critical for self-renewal and differentiation, resulting in clonal expansion of certain cells with selective proliferation under positive clonal selection pressures. In aging hematopoietic tissue, hematopoietic stem cells (HSCs) lose their capacity for self-renewal and are functionally restricted due to pathogenic mutations that result in clonal hematopoiesis (CH), or expansion and dominance of HSCs. CH is frequent and can affect over 10% of individuals >50 years old (Park, Curr Stem Cell Rep, 2018; Gondek, Hematol, 2021; Kusne, Leuk Res, 2022).

The phenomenon of somatic genomic alterations in hematopoietic cell lines, commonly referred to as clonal hematopoiesis of indeterminate potential (CHIP) or age-related clonal hematopoiesis (ARCH), has been well characterized in humans and refers to somatic alterations that occur as a result of clonal expansion of the blood system, particularly the white blood cell populations. These alterations tend to be associated with advanced age, often exhibit consistent signal over time, and are frequently found in recurrent locations in the genome (Jaiswal, Science, 2019; Jaiswal, N Eng J Med, 2014; Genovese, N Engl J Med, 2014; Gao, Nat Commun, 2021; Saiki, Nat Med, 2021). Most human patients who have these somatic alterations do not have concurrent cancer. In patients who do have concurrent cancer (specifically those with solid tumors), these alterations are typically not found in the corresponding tumor tissue. In humans, CHIP has been shown to be associated with an increased risk of developing a primary or secondary hematologic malignancy and represents an emerging biomarker for cancer risk prediction. Two population-based studies, each involving more than 10,000 people, have independently shown that the presence of CHIP alterations is associated with a roughly 10-fold higher relative risk of developing hematological cancers when compared to age-matched controls, with an absolute risk of around 0.5% per year (Jaiswal, N Eng J Med, 2014; Genovese, N Engl J Med, 2014). For this reason, leading medical centers have established “CHIP clinics” for close clinical monitoring of patients with known CHIP findings (Grisham, MSKCC, 2023; Mayo Clinic, 2023; Cleveland Clinic, 2023; Siteman Cancer Center ARCH Clinic, 2023).

### SUMMARY

Described herein are methods and compositions for the detection, of non-cancer somatic mutations in a subject. In some embodiments, the methods disclosed herein are capable of identifying such mutations where other methods known in the art were incapable of identifying these mutations.

Some embodiments provided herein relate to methods of detecting a non-cancer somatic mutation in a subject. In some embodiments, the methods include obtaining a sample from a subject, isolating genomic DNA from the sample, preparing a DNA sequencing library from the genomic DNA (gDNA), sequencing the gDNA to generate sequencing data, and detecting at least one copy number aberration in the sequencing data. In some embodiments, the at least one copy number aberration is indicative of a non-cancer somatic mutation. In some embodiments, the subject is a canine subject. In some embodiments, the sample is a whole blood sample. In some embodiments, the sequencing data is aligned to a reference genome. In some embodiments, the gDNA is extracted from white blood cells. In some embodiments, the white blood cells are present in buffy coat. In some embodiments, the methods further include isolating cell free DNA (cfDNA). In some embodiments, the cfDNA is extracted from plasma. In some embodiments, the gDNA is matched with the cfDNA. In some embodiments, an absence of non-cancer somatic mutations is detected in a cell free DNA sequencing library.

Some embodiments provided herein relate to methods of detecting a non-cancer somatic mutation in a subject. In some embodiments, the methods include isolating white blood cell (WBC) genomic DNA (gDNA) from a buffy coat sample from the subject, creating a DNA sequencing library from the WBC gDNA, generating sequencing data by sequencing the DNA sequencing library, aligning the sequencing data to a reference genome, and detecting at least one copy number aberration. In some embodiments, the at least one copy number aberration is indicative of a non-cancer somatic mutation. In some embodiments, the methods further include isolating cell-free DNA (cfDNA) from a plasma sample from the subject, creating a cfDNA sequencing library from the cfDNA, generating cell-free sequencing data by sequencing the cfDNA sequencing library, aligning the cell-free sequencing data to a reference genome, and detecting an absence of the non-cancer somatic mutations in the cell-free alignment. In some embodiments, the methods further include isolating non-buffy coat DNA from a non-buffy coat sample from the subject, creating a non-buffy coat DNA sequencing library from the non-buffy coat DNA, generating non-buffy coat sequencing data by sequencing the non-buffy coat DNA sequencing library, aligning the non-buffy coat sequencing data to a reference genome, and detecting an absence of the non-cancer somatic mutations in the non-buffy coat alignment. In some embodiments, one or more non-buffy coat somatic mutations are detected in the non-buffy coat alignment. In some embodiments, the non-buffy coat somatic mutations do not match the non-cancer somatic mutations. In some embodiments, the subject is a canine subject. In some embodiments, the buffy coat sample and the plasma sample are matched.

Some embodiments provided herein relate to methods of measuring an age related somatic alteration in a canine subject. In some embodiments, the methods include measuring a copy number variation (CNV) from white blood cell genomic DNA (WBC gDNA) obtained from a buffy sample from the canine subject, detecting an absence of CNV from cell free DNA (cfDNA) obtained from a matched plasma sample from the canine subject.

### BRIEF DESCRIPTION OF THE DRAWINGS

FIGS. 1A-1B depicts an example of a patient with WBC gDNA-specific CNVs where CNVs were identified in WBC gDNA (FIG. 1A) but not in cfDNA (FIG. 1B) in an 11-year-old neutered male mixed-breed dog with no clinical evidence of cancer.

FIGS. 2A-2C depict example CNVs identified in WBC gDNA (FIG. 2A) that are different from CNVs in cfDNA (FIG. 2B) and CNVs in tumor tissue (FIG. 2C) in a 10-year-old neutered male mixed-breed dog with hepatocellular carcinoma.

FIGS. 3A-3C depict longitudinal blood samples from a 13-year-old spayed female mixed-breed dog with no history of cancer and no suspicion of cancer showing persistence of a chromosome 25 (CFA25) gain/loss and a chromosome 36 (CFA36) gain in WBC gDNA with little to no change in the amplitude of the signal, and absence of this finding in cfDNA over a 9-month period. FIG. 3A shows WBC gDNA and cfDNA at timepoint 1; FIG. 3B shows WBC gDNA and cfDNA at timepoint 2 (3 months); FIG. 3C shows WBC gDNA and cfDNA at timepoint 3 (9 months).

FIG. 4 depicts an example plot showing normalized age distributions for dogs with and without cancer and absence of WBC gDNA specific CNVs, compared to dogs with and without cancer and presence of WBC gDNA-specific CNVs.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

Embodiments of the present disclosure relate to methods, systems, and compositions for identifying non-cancer somatic mutations in a sample from a subject by isolating buffy coat DNA from a buffy coat sample from a subject.

Embodiments relate to methods, systems, and compositions for screening subjects for their likelihood to have non-cancer somatic mutations. In some embodiments, a buffy coat sample is screened by isolating from a subject, such as a canine, a buffy coat sample and by creating a DNA sequencing library from the buffy coat DNA; generating sequencing data by sequencing the DNA sequencing library; aligning the sequencing data to a reference genome; and detecting at least one copy number aberration. In some embodiments, the at least one copy number aberration is indicative of a non-cancer somatic mutation.

The term “buffy coat” has its ordinary meaning as understood in light of the specification, and refers to the buff-colored layer resulting from the centrifugation of an anticoagulated blood sample. The buffy coat contains most of the blood cells and platelets. Typically, the buffy coat represents less than about 1% of the blood sample, with the plasma accounting for about 55%, and the red blood cell layer about 45%. The buffy coat layer is formed or “sandwiched” between the red blood cell or erythrocyte layer, which is below, and the clear plasma layer which is above. The buffy coat is useful for extracting relatively large quantities of genomic DNA, for the methods described herein.

The term “somatic,” as used herein, has its ordinary meaning as understood in light of the specification and refers to a type of genetic variant in a subject which is found in a cancer tumor, or cells derived from it. These genetic changes are thought to occur during cell divisions which lead to expansion of a tumor, but they may also have occurred in the lineage of a cancer stem cell leading up to the initiation of a tumor. A somatic mutation can give rise to cancer or other noncancerous diseases. Noncancerous somatic mutations may occur during development and may affect cell proliferation or may alter cellular function. In addition to cancer, other diseases, such as immune deficiencies, neurofibromatosis, hypertension, and others are products of somatic variations.

The term clonal hematopoiesis of indeterminate potential (CHIP) was introduced in 2015 to describe individuals that had somatic mutations known to be associated with hematological malignancies, but which were healthy and did not show any other sign or diagnostic criteria for such hematological malignancy (Steensma, Blood, 2015). Additional studies showed that clonally restricted somatic mutations are not limited to individuals with hematological cancers, but can also be detected in healthy individuals with normal blood counts (Shlush, Nature, 2014; Busque, Blood 2015). Table 1 shows the top 8 genes most frequently observed in CH and the conditions in which they are more prevalent. More than 70% of all mutations identified in CHIP occur in two genes with opposing functions—DNMT3A and TET2—leading to a similar clinical outcome: increased all-cause mortality (Jaiswal, N Eng J Med, 2014).

TABLE 1
Driver mutations involved in CH and related conditions in human.

| Gene | Function | Condition |
|---|---|---|
| DNMT3A | Epigenetic | UC, HIV, RA, Solid tumors, AML, NHL/ |
| | regulator | MM undergoing CAR-T, CVD, CHF |
| | | progression, AA, ANCA-positive vasculitis |
| TET2 | Epigenetic | RA, Solid tumors, AML, CVD, CHF |
| | regulator | progression, ANCA-positive vasculitis |
| ASXL1 | Epigenetic | ANCA-positive vasculitis, CVD, AML, AA |
| | regulator | |
| JAK2 | Signal | CVD, PV, Atherosclerosis aggravation |
| | transduction | |
| PPM1D | DNA damage | Ovarian and endometrial cancers (treated |
| | response | patients), UC, NHL/MM undergoing CAR-T |
| TP53 | DNA damage | NHL/MM undergoing CAR-T, |
| | response | chemotherapy-induced clonal expansion |
| SF3B1 | Spliceosome | Myelodysplastic Syndrome (MDS), bone |
| | | marrow ringed sideroblasts, macrocytic |
| | | anemia |
| SRSF2 | Spliceosome | MDS |

Table modified from Zerdan, Cancers (Basel), 2022.

Numerous large-scale sequencing studies have demonstrated that 10% of people >65 years old have detectable CHIP mutations, with prevalence rates increasing with age up to 20% in people older than 90 years old, depending on the study and the sequencing method used (Mckerrel, Cell Rep, 2015; Jaiswal, N Engl J Med, 2014; Genovese, N Engl J Med, 2014; Kusne, Leuk Res, 2022), CHIP mutations have also been detected in people with different conditions, including cancer patients after treatment with chemotherapy or CAR T-cell therapy (Miller, Blood Adv, 2021), people with ANCA-associated autoimmune vasculitis (Arends, Haematologica, 2020), rheumatoid arthritis (Savola, Nat Commun, 2017), ulcerative colitis (Zhang, Exp Hematol, 2019), and HIV (Dharan, Nat Med, 2021).

CHIP-positive individuals have higher risks of developing hematological malignancies and higher all-cause mortality (Xie, Nat Med, 2014; Jaiswal, N Eng J Med, 2014; Genovese, N Engl J Med, 2014). While there is a 0.5-1%/year risk of progression to develop hematologic cancer in CHIP-positive individuals, the higher all-cause mortality is mediated by an increased risk of developing cardiovascular disease, myocardial infarction, and stroke (Jaiswal, N Engl J Med, 2017; Gibson, Clin Canc Res, 2018; Min, J Intern Med, 2020; Evans, Ann Rev Path, Mechanisms of Disease, 2020).

In addition to being a well-documented risk factor for malignancy in humans, CHIP has also been associated with an increased risk for cardiovascular disease (HR 1.9; 95% CI, 1.4-2.7) (Jaiswal, N Engl J Med, 2017) and may be a risk factor for cerebrovascular events (e.g., stroke) (Mayerhover, Stroke, 2023; Steensma, Blood, 2020). Despite the growing body of literature surrounding CHIP in humans, the population level prevalence and the clinical correlates of CHIP have not been systematically studied in dogs or other species.

Apart from aging, other selection pressures known to contribute to CH include tobacco use, cancer treatments, in particular radiation and platinum-based chemotherapy, and certain inflammatory autoimmune diseases, including ulcerative colitis (UC) and rheumatoid arthritis (RA) (Arends, Haematologica, 2020; Savola, Nat Commun, 2017; Zhang, Exp Hematol, 2019; Dharan, Nat Med, 2021; Bekele, Rhem Dis Clin No Am, 2020).

While great advances in genome sequencing technologies have improved detection of CHIP mutations and the discovery of new genes associated with CHIP, further studies are needed to understand the full range of conditions associated with CHIP and finding ways to ameliorate the adverse effects of CHIP in the aging population. To achieve that, a cost-effective test that can be applied to human, but also other animal model is critical but lacking. Most of the existing technologies used for studying CHIP involves targeted deep sequencing focusing on single point mutations and is limited to testing in human.

The capabilities to detect somatic mutations such as CHIP have benefited from advances in precision medicine such as tumor tissue-based molecular testing and, most recently, from non-invasive blood testing—liquid biopsy—that relies on the analysis of cell cfDNA released by somatic cells into the bloodstream (Cohen, Science, 2018; Gale, PLOS One 2018; Plagnol, PLOS One, 2018; Jahangiri, Cancers (Basel), 2019). Following the reported detection of cancer-related alterations in circulating tumor DNA in plasma of cancer patients (Chen, Nat Med, 1996; Nawroz, Nat Med, 1996), great efforts have been devoted to developing tests to detect the presence of somatic alterations in blood, using Next Generation Sequencing (NGS) (Cohen, Science, 2018; Diehl, Nat Med, 2008; Bettegowda, Sci Transl Med, 2014; Aravanis, Cell, 2017; Liu, Ann Oncol, 2020). Recent studies have shown that CHIP is more prevalent in patients with solid cancers, with ˜30% having CH mutations in their blood (Bekel, Rhem Dis Clin NO Am, 2020). The increased use of tumor tissue- and plasma-based sequencing in recent years has helped drive clinical decision-making, and also helped drive the unintentional discovery of CH mutations from blood-based testing, ultimately increasing the prevalence of CH (Cohen, Science, 2018; Gale, PLOS One, 2018; Plagnol, PLOS One, 2018). Given the significant clinical implications that CH mutations can have on cancer patients, several institutions have established CH clinics for the treatment of patients in which genetic testing has identified CH (Ehrhart, Small An Clin Oncol, 2020; Uzuelli, Clin Chim Acta, 2009).

Recently, a blood-based liquid biopsy test using next-generation sequencing (NGS) was developed and clinically deployed for cancer detection in dogs. The clinical validation of this test involved 1100 cancer-diagnosed and presumably cancer-free client-owned dogs (Flory, PLOS One, 2022). The test has been additionally performed in thousands of dogs since it became commercially available in 2021. This recent ability to test large numbers of dogs using liquid biopsy affords an unprecedented opportunity to study the genomic profiles of a broad population of canine patients. During liquid biopsy testing in dogs, cell-free DNA (cfDNA) is extracted from plasma and genomic DNA (gDNA) is extracted from white blood cells (WBCs) present in the buffy coat; extracted DNA is then subjected to NGS to identify genomic alterations. Cell-free DNA includes DNA shed from a variety of tissues throughout the body, including tumors (if present). When genomic alterations are identified in cfDNA, this indicates the likely presence of cancer in the body. When genomic alterations are identified in gDNA, this could indicate the presence of a constitutional (germline) abnormality in the patient, including mosaicism; certain hematologic malignancies (when corresponding CNVs are also identified in cfDNA); or age-related somatic alterations (e.g., CHIP).

Most studies in humans have focused on characterizing single nucleotide variants (SNVs) in particular genes associated with CHIP, but recent reports have shown that CHIP-associated copy number variants (CNVs) are also an important risk factor for the development of leukemia and cardiovascular disease (Gao, Nat Commun, 2021; Saiki, Nat Med, 2021; Jaiswal, N Engl J Med, 2017). NGS-based liquid biopsy offers an opportunity to study if similar alterations are seen in dogs and whether they may be useful as biomarkers to predict the risk for cancer or other diseases. In some embodiments, the methods described herein use NGS. The term “Next Generation Sequencing” (NGS), as used herein, generally refers to technologies for massively parallel determination of the sequences of nucleic acid molecules, such as deoxyribonucleic acid (DNA) or ribonucleic acid (RNA) molecules. NGS was developed after, and has significantly replaced Sanger sequencing, which was considered the first generation DNA sequencing technology.

Embodiments of the methods described herein relate to the identification of recurrent CNVs in the WBC gDNA of dogs. In some embodiments, the recurrent CNVs persist with little to no change in amplitude over time and are absent from cfDNA (and from tumor tissue, in dogs with concurrent cancer). In some embodiments, the methods described herein provides early indication for the existence of age-related somatic alterations in dogs that resemble the phenomenon of CHIP previously described in humans.

Prior research on CHIP in humans involves analysis of SNVs; recent studies have also reported CHIP-associated CNVs that are likewise age-related and are associated with a higher risk of the development of leukemia in individuals with or without other existing cancer. Additionally, these studies have shown that the co-occurrence of CHIP-associated CNVs and SNVs was associated with a higher cumulative incidence of leukemia than the exclusive occurrence of either type of genomic alteration. A recent study using blood samples from cancer-diagnosed dogs found that a small percentage (4.3%; 4/93) of subjects bad genomic alterations (SNVs) in genes that have been associated with CHIP in human patients; however, no tumor tissue samples were available to confirm that these alterations did not in fact originate from the cancer itself, and no comparisons were made between WBC-derived gDNA and plasma-derived cfDNA.

Embodiments of the present disclosure relate to analysis of WBC gDNA-specific CNVs in canine subjects. Some embodiments provided herein relate to evaluation of the prevalence and distribution of WBC gDNA-specific SNVs in large populations of dogs. In some embodiments, the methods relate to evaluation of the interplay between multiple types of CHIP-associated genomic alterations and clinical outcomes.

Embodiments of the present disclosure relate to measurement of the population-level frequency of age-related somatic copy number aberrations in the WBC gDNA of dogs and characterization of the type of alterations observed in these subjects.

“Copy number aberration” or CNA, as used herein has its usual meaning as understood in light of the specification, and refers to a change in the number of copies of a particular genetic sequence or component within an individual genome and can range from losses (deletions) of one or more copies of the genetic component to gains of numerous additional copies of the genetic component (amplifications). One type of CNA is an “aneuploidy”, which generally refers to an abnormal number of whole chromosomes. Typically, aneuploidy may result from a genetic imbalance resulting from cancer or other diseases. In some embodiments, aneuploidies results in either three (“trisomy”) or only one (“monosomy”) chromosome. In some embodiments, measuring aneuploidy may be used in the context of cancer diagnostics as described above.

The sequencing of the DNA sequencing library can be performed through any method recognized by those of skill in the art, including, for example, targeted or genome-wide sequencing. Other non-limiting examples include methods using nanopores, emulsion, and “sequencing by binding” cycled sequencing methods. As used herein a “nucleic acid library” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). A DNA sequencing library is a sample of genomic DNA fragments purified from a particular biological source representing the entire genome of said biological source. In a DNA sequencing library, the genomic DNA fragments may be 3′- and 5′-ligated to primers and adapter sequences for further analysis of the genomic DNA fragments (e.g., sequencing analysis).

For example, the preparation of a DNA library for sequencing may start with the fragmentation of the DNA sample, which was purified from a particular biological source. The fragmentation defines the molecule entry points for the sequencing reads. In a next step. DNA ends may be enzymatically repaired and adenine (A) may be added to the 3′ ends of the DNA fragments. The (A)-tailed DNA fragments may then be amplified as templates to ligate double-strand, partially complementary adapters to the DNA fragments. The DNA library may then be size-selected and amplified to improve the quality of sequence reads. The amplification reaction introduces specific PCR primers to the adapter sequences that are required for sequencing n the flow cell.

In some embodiments, non-cancer somatic mutations are screened by isolating cell-free DNA from a plasma sample from the subject creating a cell-free DNA sequencing library from the cell-free DNA; generating cell-free sequencing data by sequencing the cell-free DNA sequencing library; aligning the cell-free sequencing data to a reference genome; and detecting an absence of the non-cancer somatic mutations in the cell-free alignment. Models are derived from the fragment size distribution profile of at least one fragment. “Fragment distribution” as used herein has its usual meaning as understood by those skilled in the art and thus refers to the length, sequence, fragmentation, and other distribution properties of an at least one DNA fragment taken from a cfDNA sample. A “fragment size distribution” is understood as a fragment distribution focusing on the size of fragments, including length or fragmentation. As disclosed herein, a model can be formed for the subject suspected of having a cancer or a tumor, as well as a model for one or more healthy subjects. These models can then be compared to one another to monitor for significant differences. Non-limiting examples of models include summary statistics, the number and shape of nucleosomal peaks, the proportion of fragments longer or shorter than a certain threshold, the proportion of fragments in certain intervals, the approximation of the data with statistical distributions, and discriminatory learning methods, such as support vector machines or neural networks. Non-limiting examples of detectable differences include the location of the peaks (mode), the height of the peaks (weight), the spread of the peaks (scale), the proportion of fragments longer or shorter than a certain threshold, the amplitude of oscillations, the overall shape of the fragment size distribution, Principal Component values, and Kullback-Leibler (KL) divergence between two models. In some embodiments, a statistically significant difference between the fragment size distribution in the subject suspected of having a cancer or tumor and the fragment size distribution in the one or more healthy subjects indicates the presence of a cancer or tumor. In some embodiments, a non-statistically significant difference between the fragment size distribution in the subject suspected of having a cancer or tumor and the fragment size distribution in the one or more healthy subjects indicates the lack of presence of a cancer or tumor.

As used herein, the term “sequence alignment” or “sequence mapping” refers to a way of arranging the sequences of DNA or RNA, relative to one another so as to identify regions of interest, such as regions of similarity or regions of variation. Such sequences may be a consequence of functional, structural, or evolutionary relationships between the sequences. Aligned sequences of nucleotides are typically represented as rows within a matrix. Well-known algorithms for sequence alignments are for instance Needleman-Wunsch algorithm, Smith-Waterman algorithm or Waterman-Eggert algorithm or Burrows-Wheeler transform. Well-known tools for sequence alignments are for instance BLAST, BLAT, WMBOSS, Clustal, BWA, Bowtie.

In some embodiments, non-cancer somatic mutations are screened by isolating non-buffy coat DNA from a non-buffy coat sample from the subject creating a cell-free DNA sequencing library from the cell-free DNA; generating a non-buffy coat sequencing data by sequencing the non-buffy coat DNA sequencing library; aligning the non-buffy coat sequencing data to a reference genome; and detecting an absence of the non-cancer somatic mutations in the cell-free alignment.

In some embodiments, one or more non-buffy coat somatic mutations are detected in the non-buffy coat alignment. In some embodiments, the non-buffy coat somatic mutations do not match the non-cancer somatic mutations.

A variety of ways exist for determining the fragment size distribution of the cfDNA within a subject. In one embodiment a blood sample is taken from a subject. Circulating free DNA (cfDNA) from the blood is obtained. In some embodiments, the blood sample includes circulating tumor DNA (ctDNA), The cfDNA is isolated by removing blood cells from the sample so that only cfDNA remains in the sample. In some embodiments, a set of random PCR primers for whole genome sequencing are added to the sample to amplify the fragments while preserving their original fragment length within the sample.

Polymerase is then added to the mixture, so the primers are extended through the full length of each fragment. The amplified fragments may include sequencing ends which are formatted to be used within a Next Generation Sequencing (NGS) system to identify the nucleotide sequences in the fragments in one embodiment.

Methods and compositions provided herein improve the detection, diagnosis, staging, screening, treatment, and management of cancer in subjects, particularly in humans, mammals, and other types of subjects. As mentioned above, embodiments include identifying the fragment distribution of cfDNA circulating biological fluids, such as blood. In one embodiment, the nucleic acid sequence elements are found in circulating tumor DNA in the blood. In some embodiments, the nucleic acid sequence elements may be found in cell-free DNA, in saliva, or urine.

As used herein, “detecting” with respect to measuring a cancer or tumor includes the use of an instrument used to observe and record a signal corresponding to a level or measurement of cancer, or materials required to generate such a signal. In various embodiments, the detecting includes any suitable method, including amplification, sequencing, arrays, fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

Some embodiments provided herein relate to kits. In some embodiments, the kits are for determining cancer in a subject. In some embodiments, the kits include whole genome sequencing primers for amplifying cfDNA in a biological sample from a subject, and a polymerase for amplifying the primers.

It should be realized that the analysis described herein may be part of a larger diagnostic suite used to determine a subject's overall health. For example, the analysis of fragment size distributions of cfDNA in a subject may be used simultaneously or sequentially with other methods for detection, diagnosis, staging, screening, monitoring, treatment, and management of cancer including additional genetic variance analysis. These procedures may be useful to detect a variety of cancers, including leukemia, squamous cell carcinoma, feline mammary cancer, mast cell tumors, bladder cancer, osteosarcoma, hemangiosarcoma or a variety of other cancers afflicting subjects.

In some embodiments, the methods include obtaining or having obtained a biological sample from a subject that is. In some embodiments, the sample is a liquid biopsy sample, such as a blood sample. In some embodiments, the sample includes cfDNA. In some embodiments, the sample is provided in an amount of less than 10 mL, such as 10 mL, 9 mL, 8 mL, 7 mL, 6, mL, 5 mL, 4 mL, 3 mL 2 mL, 1 mL, 500 μL, 250 μL, 100 μL or an amount within a range defined by any two of the aforementioned values. In some embodiments, the sample includes DNA in an amount of less than or equal to 10 μg, such as 10 μg, 5 μg, 1 μg, 500 ng, 100 ng, 50 ng, 10 ng, 5 ng, 1 ng, 500 pg, 100 pg, 50 pg, 10 pg, 9, pg, 8 pg, 7 pg, 6 pg, 5 pg, 4 pg, 3 pg, 2 pg, or 1 pg, or in an amount within a range defined by any two of the aforementioned values. In some embodiments, the method includes purifying the DNA from the sample. Purifying the DNA may be accomplished using DNA purification techniques, including, for example extraction techniques, precipitations, chromatography, bead-based methods, or commercially available kits for DNA purification. In some embodiments, the methods can be used to determine the probable cancer type or cancer tissue of origin based on one or more of the fragment size distribution features.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

As used herein, “a” or “an” can mean one or more than one.

As used herein, the term “about” or “approximately” has its usual meaning as understood by those skilled in the art and thus indicates that a value includes the inherent variation of error for the method being employed to determine a value, or the variation that exists among multiple determinations.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “function” and “functional” as used herein have their plain and ordinary meaning as understood in light of the specification, and refer to a biological, enzymatic, or therapeutic function.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 81, 82, 83, 84, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, or living cells which make up these living organisms, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

As used herein, “nucleic acid”, “nucleic acid molecule”, or “nucleotide” refers to polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which include naturally occurring or modified nucleic acid bases attached to a polyamide backbone. Nucleic acids can be either single stranded or double stranded.

The terms “peptide”, “polypeptide”, and “protein” as used herein have their plain and ordinary meaning as understood in light of the specification and refer to macromolecules made up of of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The terms “DNA fragment” and “nucleic acid fragment” have their ordinary meaning as understood by those of skill in the art and refer to a polynucleotide sequence obtained from a genome at any point along the genome and encompassing any sequence of nucleotides.

The term “fragment size distribution” has its ordinary meaning as understood by those of skill in the art, and refers to information regarding one or more of: the total number of nucleic acid fragments present in a sample, the size of one or more nucleic acid fragments in the sample, the absolute or relative abundance levels of nucleic acid fragments of a specific size or size range, and the absolute or relative abundance levels of nucleic acid fragments of different size present in the sample.

The term “fragment size” has its ordinary meaning as understood by those of skill in the art, and as used herein in reference to a nucleic acid molecule, refers to the number of base pairs of the nucleic acid, and denotes the length of the molecule.

The term “gene” as used herein have their plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene includes transcribed sequences that encode for a protein, polypeptide, or peptide. In keeping with the terminology described herein, an “isolated gene” may include transcribed nucleic acid(s), regulatory sequences, coding sequences, or the like, isolated substantially away from other such sequences, such as other naturally occurring genes, regulatory sequences, polypeptide, or peptide encoding sequences, etc. In this respect, the term “gene” is used for simplicity to refer to a nucleic acid including a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express or may be adapted to express using nucleic acid manipulation technology, proteins, polypeptides, domains, peptides, fusion proteins, mutants and/or such like.

The terms “cancer” and “cancerous” have their ordinary meaning as understood in light of the specification and refer to or describe the physiological condition in animals that is typically characterized by unregulated cell growth. A “tumor” includes one or more cancerous cells. In some embodiments, the tumor is a solid tumor. There are several main types of cancer. Carcinoma is a cancer that originates from epithelial cells, for example skin cells or lining of intestinal tract. Sarcoma is a cancer that originates from mesenchymal cells, for example bone, cartilage, fat, muscle, blood vessels, or other connective or supportive tissue. Leukemia is a cancer that originates in hematopoietic cells, such as the bone marrow, and causes large numbers of abnormal blood cells to be produced and enter the blood. Lymphoma and multiple myeloma are cancers that originate in the lymphoid cells of lymph nodes. Central nervous system cancers are cancers that originate in the central nervous system and spinal cord.

As used herein, the phrase “allele” or “allelic variant” has its ordinary meaning as understood in light of the specification and refers to a variant of a locus or gene. In some embodiments, a particular allele of a locus or gene is associated with a particular phenotype, for example, altered risk of developing a disease or condition, likelihood of progressing to a particular disease or condition stage, amenability to particular therapeutics, susceptibility to infection, immune function, etc.

As used herein, the term “amplification” has its ordinary meaning as understood in light of the specification and refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100-fold difference). Amplification by asymmetric PCR is generally linear. A skilled artisan will understand that different amplification methods may be used together.

As used herein, “amplicon” has its ordinary meaning as understood in light of the specification and refers to the nucleic acid sequence that will be amplified as well as the resulting nucleic acid polymer of an amplification reaction. An amplicon can be formed artificially, such as through polymerase chain reactions (PCR) or ligase chain reactions (LCR), or naturally through gene duplication.

The terms “individual”, “subject”, “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

As used herein, the term “liquid biopsy” has its ordinary meaning as understood in light of the specification and refers to the collection of a sample and the testing the sample, wherein the sample is non-solid biological tissue such as blood.

As used herein, the term “cfDNA” has its ordinary meaning as understood light of the specification, and refers to circulating cell free DNA, which includes DNA fragments released to the blood plasma. cfDNA can include circulating tumor deoxyribonucleic acid (ctDNA).

As used herein, the term “ctDNA” has its ordinary meaning as understood in light of the specification, and refers to circulating tumor DNA, which includes a tumor-derived fragmented DNA in the bloodstream that is not associated with cells.

Some embodiments provided herein are described in the following enumerated alternatives.
1. A method of detecting a non-cancer somatic mutation in a subject, the method comprising: obtaining a sample from a subject; isolating genomic DNA from the sample; preparing a DNA sequencing library from the genomic DNA (gDNA); sequencing the gDNA to generate sequencing data; and detecting at least one copy number aberration in the sequencing data, wherein the at least one copy number aberration is indicative of a non-cancer somatic mutation.
2. The method of alternative 1, wherein the subject is a canine subject.
3. The method of any one of alternatives 1-2, wherein the sample is a whole blood sample.
4. The method of any one of alternatives 1-3, wherein the sequencing data is aligned to a reference genome.
5. The method of any one of alternatives 1-4, wherein the gDNA is extracted from white blood cells.
6. The method of alternative 5, wherein the white blood cells are present in buffy coat.
7. The method of any one of alternatives 1-6, further comprising isolating cell free DNA (cfDNA).
8. The method of alternative 7, wherein the cfDNA is extracted from plasma.
9. The method of any one of alternatives 7-8, wherein the gDNA is matched with the cfDNA.
10. The method of any one of alternatives 7-9, wherein an absence of non-cancer somatic mutations is detected in a cell free DNA sequencing library.
11. A method of detecting a non-cancer somatic mutation in a subject, the method comprising: isolating white blood cell (WBC) genomic DNA (gDNA) from a buffy coat sample from the subject; creating a DNA sequencing library from the WBC gDNA; generating sequencing data by sequencing the DNA sequencing library; aligning the sequencing data to a reference genome; and detecting at least one copy number aberration, wherein the at least one copy number aberration is indicative of a non-cancer somatic mutation.
12. The method of alternative 11, further comprising: isolating cell-free DNA (cfDNA) from a plasma sample from the subject, creating a cfDNA sequencing library from the cfDNA; generating cell-free sequencing data by sequencing the cfDNA sequencing library; aligning the cell-free sequencing data to a reference genome; and detecting an absence of the non-cancer somatic mutations in the cell-free alignment.
13. The method of any one of alternatives 11-12, further comprising: isolating non-buffy coat DNA from a non-buffy coat sample from the subject; creating a non-buffy coat DNA sequencing library from the non-buffy coat DNA; generating non-buffy coat sequencing data by sequencing the non-buffy coat DNA sequencing library; aligning the non-buffy coat sequencing data to a reference genome; and detecting an absence of the non-cancer somatic mutations in the non-buffy coat alignment.
14. The method of alternative 13, wherein one or more non-buffy coat somatic mutations are detected in the non-buffy coat alignment, and wherein the non-buffy coat somatic mutations do not match the non-cancer somatic mutations.
15. The method of any one of alternatives 11-14, wherein the subject is a canine subject.
16. The method of any one of alternatives 11-15, wherein the buffy coat sample and the plasma sample are matched.
17. A method of measuring an age related somatic alteration in a canine subject, comprising: measuring a copy number variation (CNV) from white blood cell genomic DNA (WBC gDNA) obtained from a buffy sample from the canine subject; and detecting an absence of CNV from cell free DNA (cfDNA) obtained from a matched plasma sample form the canine subject.

### EXAMPLES

Embodiments of the present disclosure are further defined in the following Examples. It should be understood that these Examples are given by way of illustration only. From the above discussion and these Examples, one skilled in the art can ascertain the essential characteristics described herein, and without departing from the spirit and scope thereof, can make various changes and modifications of the embodiments described herein to adapt it to various usages and conditions. Thus, various modifications of the embodiments described herein, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

Isolating Buffy Coat DNA from a Sample

Blood samples were processed with a double-centrifugation protocol to separate plasma from the buffy coat (white blood cells). Cell-free DNA was extracted from plasma using a proprietary bead-based chemistry protocol optimized to maximize cell-free DNA yield. DNA from buffy coat was extracted using standard DNA extraction methods such as QIAamp DNA Mini Blood Kit (Qiagen). Libraries were prepared by incorporating universal adapters and barcodes into sample DNA via ligation and universal PCR amplification. Amplified libraries were subjected to genome-wide sequencing for CNV analysis. All libraries were sequenced using an Illumina NovaSeq 6000.

### Example 2

### Copy Number Variant Analysis

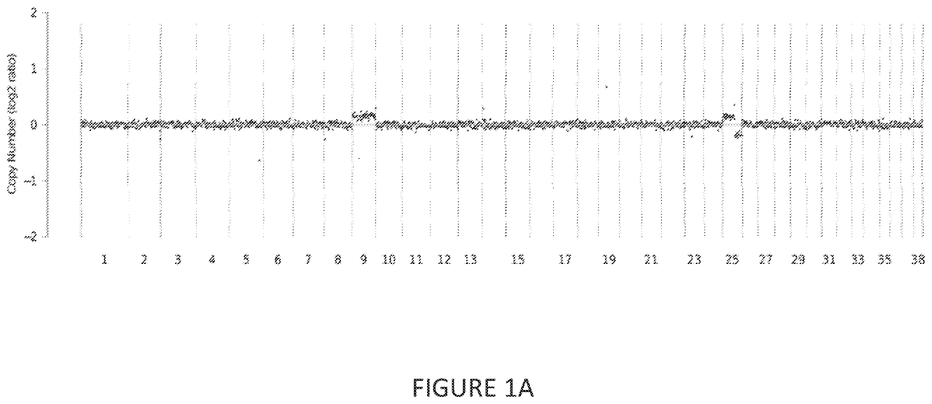

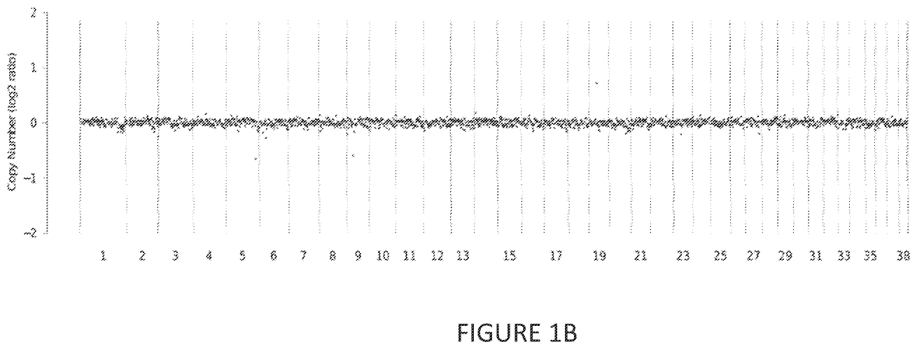

When analyzing copy number variants (CNVs) from the CANDID study and from a commercial cohort (N=2925), CNVs unique to the gDNA sample (for example, CHIP) were identified in 139 dogs (4.8%). FIGS. 1A and 1B show examples of CHIP CNVs in the gDNA that are either distinct from cancer-derived somatic CNVs in the cfDNA (FIG. 1A) or that are clearly visible despite no evidence of signal in cfDNA in a presumed cancer-free subject (FIG. 1B). A number of these CNVs were recurrent across patients, consistent with observations of recurrent genes affected by mutations in human CHIP.

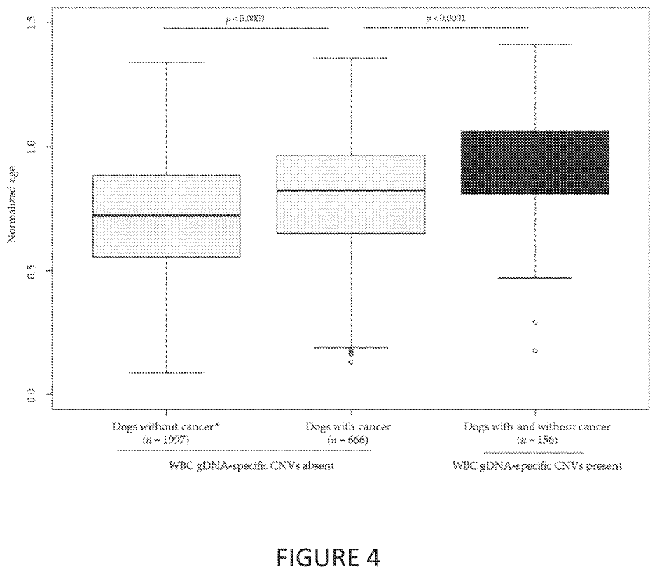

In 50 of the 139 total affected patients, multiple plasma samples from follow-up timepoints were available for analysis, and among this group, 37 showed persistent CHIP signal across timepoints, with no visible increase in signal amplitude suggestive of cancer progression. Of the 139 dogs with CHIP, matched tumor tissue was available and showed a copy number signature in 11 patients; the genomic profiles of the tumor tissue and gDNA in those patients were uncorrelated 10 of these cases, corresponding to a tissue confirmation rate of 9.1%. In contrast, for subjects with cfDNA CNVs, matched tumor tissue was available and showed a copy number signature in 94 subjects, and in this group the tissue confirmation rate was 92.6% ( 87/94). These results suggest that the concomitant presence of gDNA-specific somatic alterations was incidental to the patient's cancer. Finally, the normalized age (required to account for breed specific differences in expected lifespan; calculated as age normalized to expected lifespan, which is a linear function of weight) of subjects with CHIP was compared to the normalized age of subjects with confirmed cancer absent CHIP signal, as well as to the normalized age of subjects in a high-risk screening population with no known cancer and no evidence of CHIP. As shown in FIG. 4, the age of patients with CHIP was significantly older than the ages of either of the other cohorts (p<0.001 for both comparisons).

### Example 3

Blood-based liquid biopsy test using next-generation sequencing (NGS) was developed and clinically deployed for cancer detection in dogs. During liquid biopsy testing in dogs, cell-free DNA (cfDNA) was extracted from plasma and genomic DNA (gDNA) was extracted from white blood cells (WBCs) present in the buffy coat; extracted DNA was subjected to NGS to identify genomic alterations. Cell-free DNA includes DNA shed from a variety of tissues throughout the body, including tumors (if present). When genomic alterations are identified in cfDNA, this indicates the likely presence of cancer in the body. When genomic alterations are identified in gDNA, this could indicate the presence of a constitutional (germline) abnormality in the patient, including mosaicism; certain hematologic malignancies (when corresponding CNVs are also identified in cfDNA); or age-related somatic alterations (e.g., CHIP).

Most studies in humans have focused on characterizing single nucleotide variants (SNVs) in particular genes associated with CHIP, but recent reports have shown that CHIP-associated copy number variants (CNVs) are also an important risk factor for the development of leukemia and cardiovascular disease (Gao, Nat Commun, 2021; Saiki, Nat Med, 2021; Jaiswal, N Engl J Med, 2017). NGS-based liquid biopsy offers an opportunity to study whether similar alterations are seen in dogs and whether they may be useful as biomarkers to predict the risk for cancer or other diseases.

A total of 4870 client-owned dogs were evaluated in this example, as detailed in Table 2. Of this total, whole blood samples from 3595 dogs with and without a clinical suspicion of cancer were submitted by the dog's veterinarian for commercial liquid biopsy testing at the PetDx laboratory in La Jolla, CA (“clinical cohort”); whole blood samples from 1275 dogs with and without a diagnosis of cancer were obtained as part of a larger research collection to support the clinical validation of the NGS-based liquid biopsy test (“research cohort”) (Flory, PLos One, 2022). All research subjects were enrolled under protocols that received Institutional Animal Care and Use Committee (IACUC) or site-specific ethics approval (Flory, PLos One, 2022). All methods were performed in accordance with the relevant guidelines and regulations and follow the recommendations in the ARRIVE guidelines. All research subjects were client-owned, and written informed consent was obtained from all owners. In a subset of cancer-diagnosed patients from the research cohort, matched tumor tissue samples were also available for analysis. Additionally, a subset of patients across both the clinical and the research cohorts submitted whole blood samples at multiple timepoints, allowing for longitudinal monitoring of genomic alterations.

TABLE 2
Demographic of Dogs in the Clinical and Research Cohorts

| | |
|---|---|
| Clinical Cohort | Research Cohort |
| (n = 3595) N (%) | (n = 1275) N (%) |

Cancer Status

| Cancer suspected | 962 | (26.8%) | — |
|---|---|---|---|
| Cancer not suspected | 2399 | (66.7%) | — |
| Cancer suspicion not | 234 | (6.5%) | — |
| provided | | | |

| | | | |
|---|---|---|---|
| Cancer-diagnosed | — | 569 | (44.6%) |
| Presumably cancer-free | — | 706 | (55.4%) |

Sex

| Male | 1797 | (50.0%) | 665 | (52.2%) |
|---|---|---|---|---|
| Neutered | 1581 | | 572 | |
| Intact | 204 | | 92 | |
| Not Provided | 12 | | 1 | |
| Female | 1750 | (48.7%) | 610 | (47.8%) |
| Spayed | 1608 | | 553 | |
| Intact | 96 | | 56 | |
| Not Provided | 46 | | 1 | |
| Sex not provided | 48 | (1.3%) | 0 | (0%) |

Age (years)

| | | | | |
|---|---|---|---|---|
| Mean (SD) | 9.1 | (3.1) | 7.2 | (3.7) |

Weight (kg)

| | | | | |
|---|---|---|---|---|
| Mean (SD) | 25.8 | (13.5)* | 28.3 | (12.0) |

Breed Classification

| | | | | |
|---|---|---|---|---|
| Purebred | 2198 | (61.1%) | 630 | (49.4%) |
| Mixed-breed or unknown | 1397 | (38.9%) | 645 | (50.6%) |

*Weight was available for 3144 dogs in the clinical cohort.

Whole blood samples were collected from each patient; cfDNA was extracted from plasma, and gDNA was extracted from WBCs present in the buffy coat; in the subset of patients with matched tumor tissue available, DNA was also extracted from tissue (Flory, PLOS One, 2022; Kruglyak, Front Vet Sci, 2021). All extracted DNA specimens were subjected to library preparation and NGS as previously described (Flory, PLOS One, 2022). Sequencing data were analyzed using an internally developed bioinformatics pipeline to determine the presence of genomic alterations.

This example focused on a large cohort of canine patients in which specific CNVs were identified in WBC gDNA but were absent in matched cfDNA samples. These findings are referred to herein as “WBC gDNA-specific CNVs”, and one example is shown in FIGS. 1A and 1B. A subset of patients with WBC gDNA-specific CNVs also had concurrent CNVs identified in cfDNA (and/or tissue, when available) but those CNVs were different from the CNVs identified in WBC gDNA.

A subset of dogs with WBC gDNA-specific CNVs had clinical evaluations performed to determine the presence of cancer. Though the components of each evaluation varied, they typically included a thorough physical examination, laboratory workup (CBC, chemistry panel, and urinalysis), imaging (thoracic radiographs and/or abdominal ultrasound), and tissue sampling (via fine needle aspiration or biopsy) of observed masses, lesions, and/or enlarged lymph nodes.

When the clinical evaluation determined that cancer was present, a definitive or presumptive cancer diagnosis was assigned, as shown in Table 3. “Definitive” diagnoses were those in which cancer was confirmed via tissue-based testing (cytology or histopathology). “Presumptive” diagnoses were based on imaging, direct visualization/exam, or a strong suspicion from cytology or histopathology.

TABLE 3
Disposition of Dogs in the Clinical and Research Cohorts
Based on the Presence or Absence of gDNA-Specific CNVs
and Concurrent CNVs in Plasma-Derived cfDNA.

| | Clinical | Research |
|---|---|---|
| | Cohort | Cohort * |
| | (n = 3595) N; | (n = 1275) N; |
| | % [95% CI] | % [95% CI] |
| gDNA-specific CNVs present | 107; 3.0 | 19; 1.5 |
| without concurrent cfDNA CNVs | [2.5, 3.6] | [0.9, 2.4] |
| Cancer evaluation performed | 28 | 14 |
| Cancer | 9 | 14 |
| No cancer | 19 | 0 |
| No additional cancer evaluation | 79 | 5 |
| following liquid biopsy | | |
| gDNA-specific CNVs present with | 22; 0.6 | 16; 1.3 |
| concurrent cfDNA CNVs | [0.4, 0.9] | [0.7, 2.1] |
| Cancer evaluation performed | 11 | 15 |
| Cancer | 11 | 15 |
| No cancer | 0 | 0 |
| No additional cancer evaluation | 11 | 1 |
| following liquid biopsy | | |
| gDNA-specific CNVs absent | 3466; 96.4 | 1240; 97.3 |
| | [95.8, 97.0] | [96.2, 98.0] |
| Cancer evaluation performed | 422 | 549 |
| Cancer | 124 | 542 |
| No cancer | 298 | 7 |
| No additional cancer evaluation | 3044 | 691 |
| following liquid biopsy | | |

* The term “cancer evaluation” in the context of this example refers to a thorough workup performed when cancer is suspected (because of liquid biopsy results and/or because of the patient's clinical presentation); the components of this workup are described herein. Cancer-free dogs in the research cohort had a clinical history and physical exam at the time of study enrollment.

To compare the age profiles of adult dogs with and without WBC gDNA-specific CNVs, a “normalized age” was calculated relative to expected lifespan, where expected lifespan was calculated as a function of weight according to the equation: lifespan=13.987−0.035×weight (Beuchat, 2022). p-values were calculated using a two-sided t-test. Analyses were performed using R (version 4.0.5).

The population of 4870 client-owned dogs included 3595 dogs from the clinical cohort and 1275 dogs from the research cohort (Table 3).

In the clinical cohort, cancer status was unknown at the time of sample submission. Samples for 962 dogs were submitted due to a clinical suspicion of cancer (liquid biopsy was used as an aid in diagnosis); samples for 2399 dogs were submitted with no reported suspicion of cancer (liquid biopsy was used as a screening test); and samples for 234 dogs were submitted without documentation regarding cancer suspicion. The average age of dogs in the clinical cohort was 9.09 years (SD=3.06; n=3595) and the average weight was 25.80 kg (SD=13.48; n=3144).

In the research cohort, 569 dogs had a definitive diagnosis of cancer and 706 dogs were presumably cancer-free. The average age of dogs in the research cohort was 7.17 years (SD=3.68; n=1275) and the average weight was 28.28 kg (SD=12.04; n=1275) A wide range of purebred and mixed-breed dogs were represented in both cohorts.

WBC gDNA-specific CNVs were identified in 164 of the 4870 (3.4%; 95%) CI: 2.9-3.9) dogs in the overall study population: 129 from the clinical cohort and 35 from the research cohort. For 126 of these dogs (107 from the clinical cohort and 19 from the research cohort), CNVs were identified only in the WBC gDNA, and no CNVs of any kind were observed in plasma-derived cfDNA.

Clinical cancer evaluations were performed in 42 of the 126 dogs with WBC gDNA specific CNVs: 28 from the clinical cohort and 14 from the research cohort.

Of the 28 patients in the clinical cohort, 18 had samples submitted with no suspicion of cancer at the time of blood draw (for example, the test was used for cancer screening). Within this group, 78% ( 14/18; 95% CI: 51.9-92.6) had no evidence of cancer following clinical evaluation, while 22% ( 4/18; 95% CI: 7.4-48.1) received a definitive or presumptive cancer diagnosis. The four cancer diagnoses in the screening patients included splenic stromal sarcoma (definitive, diagnosed via histopathology), a splenic mass that was suspected to be cancer on imaging but did not have tissue testing for confirmation (presumptive), a hepatic mass (presumptive), and an adrenal gland tumor (presumptive).

The remaining 10 out of 28 patients in the clinical cohort had samples submitted for testing due to suspicion of cancer (for example, the test was used as an aid in diagnosis). Within this group, 50% ( 5/10; 95% CI: 20.1-79.9) had no evidence of cancer following clinical evaluation, while 50% ( 5/10; 95% CI: 20.1-79.9) received a definitive or presumptive cancer diagnosis. The 5 cancer diagnoses in the aid-in-diagnosis patients included, histiocytic sarcoma (definitive), a mixed germ cell-sex cord stromal tumor (definitive), two bone tumors (both presumptive), and an adrenal gland tumor (presumptive).

All 14 dogs whose samples were submitted as part of the research collection had a definitive diagnosis of cancer at the time of blood collection. The cancer diagnoses in these dogs were as follows: lymphoma/acute lymphoid leukemia (4 cases), osteosarcoma (3 cases), mast cell tumor (2 cases), hemangiosarcoma, anal sac adenocarcinoma, chronic lymphoid leukemia, soft tissue sarcoma, and transitional cell carcinoma.

There were 38 dogs with CNVs identified in plasma-derived cfDNA in addition to WBC gDNA-specific CNVs (22 from the clinical cohort and 16 from the research cohort); 26 of these dogs (11 from the clinical cohort and 15 from the research cohort) bad clinical cancer evaluations performed. Among those in the research cohort, all were among the 569 dogs who were enrolled with a definitive diagnosis of cancer. The cancer confirmation rate for these 26 dogs was 100% (26/26; 95% CI: 84.0-100), demonstrating that observations of somatic CNVs in the plasma cfDNA are highly predictive of concurrent cancer.

Tumor tissue samples (from the same collection timepoint as the blood samples with WBC gDNA-specific findings) were available for testing by NGS from 11 of the 35 cancer diagnosed dogs in the research cohort. The CNVs observed in gDNA were not present in the matched tumor tissue in 91% of cases ( 10/11). In one case, the same CNVs observed in WBC gDNA were present in matched tissue (from a fine needle aspirate of the left mandibular lymph node); this subject had a definitive diagnosis of T-zone lymphoma (stage IIa).

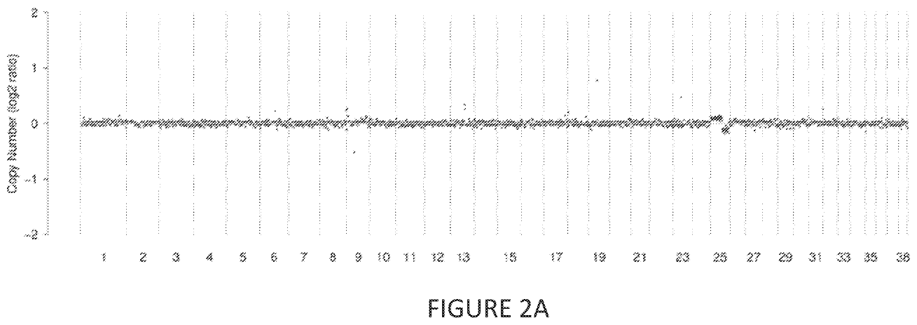

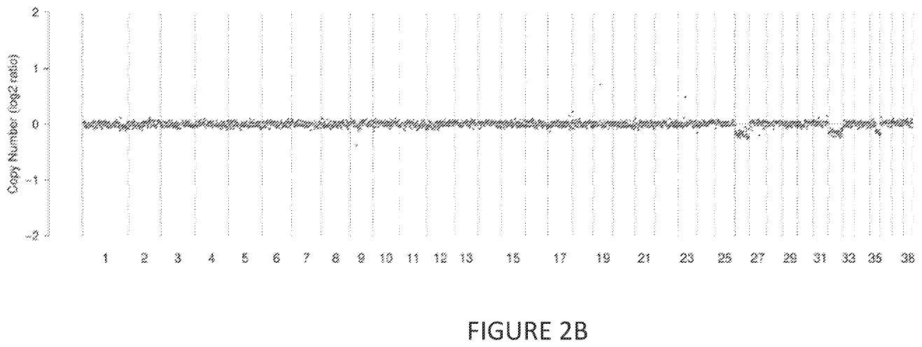

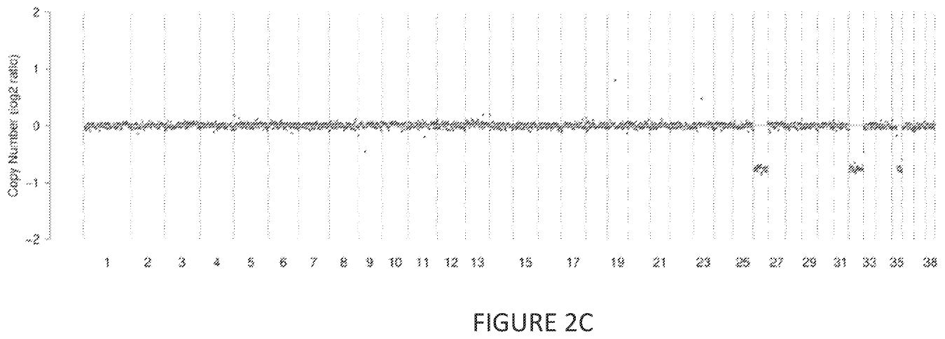

Tumor tissue samples (from the same collection timepoint as the blood samples with cfDNA-specific CNV findings) were available for testing by NGS from 105 cancer diagnosed dogs in the research cohort. In contrast to WBC gDNA-specific CNVs, the CNVs observed in cfDNA did match the CNVs found in tumor tissue in the vast majority of cases (92%; 97/105). FIGS. 2A-2C show the matched CNV profiles from one subject with CNVs present in tissue, cfDNA, and gDNA. The WBC gDNA-specific CNVs on CFA25 (canine chromosome 25; FIG. 2A) are not visible in the tumor tissue (FIG. 2C); however, the cfDNA-specific CNVs on CFA26, CFA32, and CFA35 (FIG. 2B) are clearly visible in the tumor tissue.

These results suggest that WBC gDNA-specific CNVs, when present, are typically coincidental (but not biologically related) to the patient's cancer, while cfDNA-specific CNVs are typically derived from the patient's cancer.

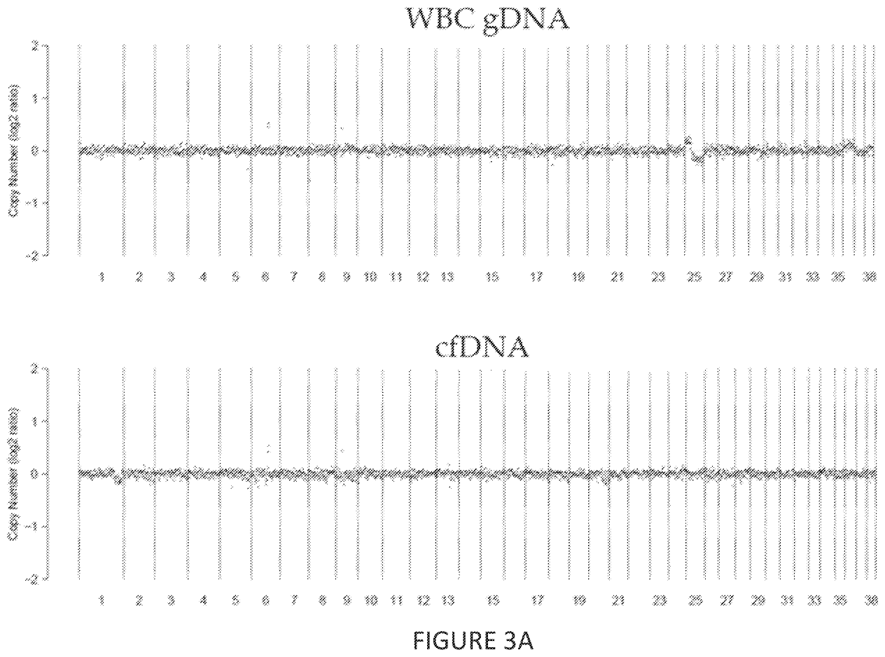

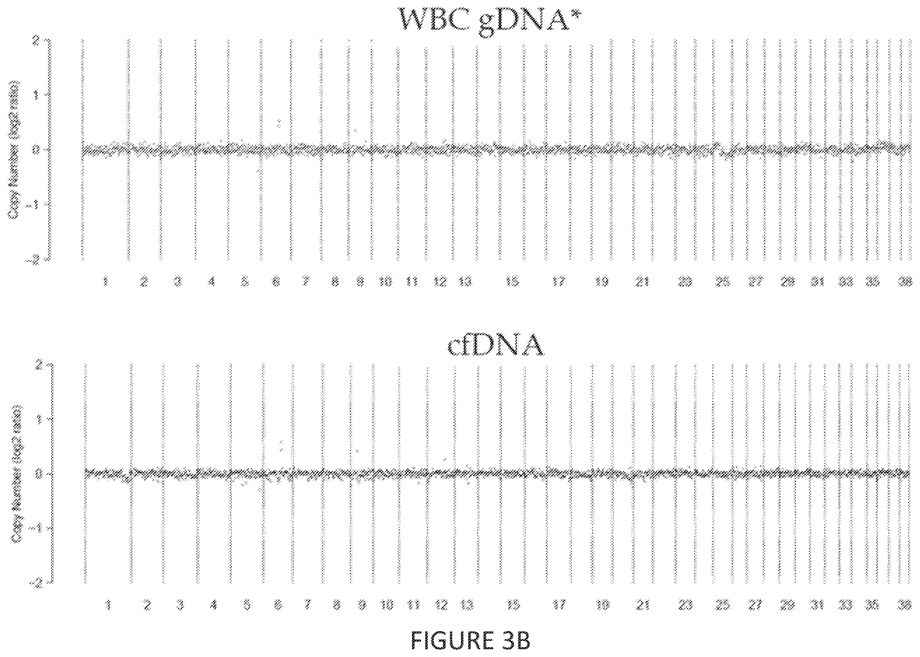

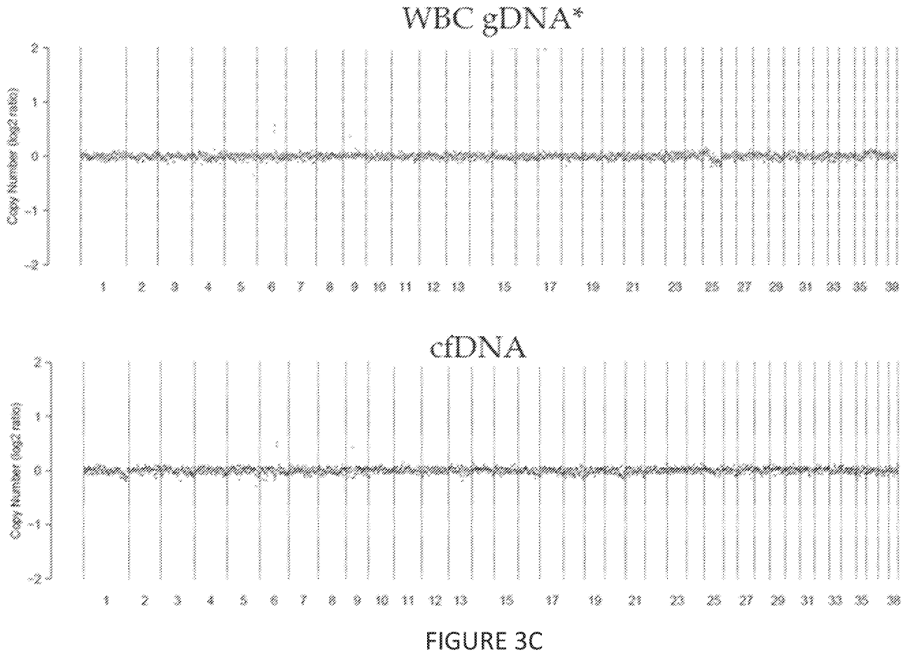

Longitudinal blood samples were available for a total of 76 patients with WBC gDNA-specific findings. Seventy-one percent of patients ( 54/76; 95% CI: 60.0-80.0) showed persistence of the gDNA CNVs over time. FIGS. 3A-3C show CNV profiles from one of these patients, with persistent WBC gDNA-specific CNVs in CFA25 and CFA36 in three consecutive timepoints spanning 9 months. In the remaining 29% ( 22/76; 95% CI: 19.4-40.7) of cases, the gDNA CNVs were not persistent across all subsequent timepoints.

Several WBC gDNA-specific CNVs were recurrent across patients; the most common CNV was a partial loss of chromosome 25, typically accompanied by a partial gain on the same chromosome (Table 4; FIGS. 1A and 2A). This pattern is consistent with human literature, where CHIP has been reported at recurrent locations in the genome (Gao, Nat Commun, 2021; Saiki, Nat Med, 2021; Takahashi, Blood Adv, 2017).

TABLE 4
Recurrent Copy Number Variants Identified
in WBC gDNA and Their Frequency in the Population
of Dogs with WBC gDNA-Specific CNVs

| | | Number of | % Dogs with WBC gDNA- |
|---|---|---|---|
| | Gain/ | Dogs with | Specific CNVs with This |
| Chromosome | Loss | this Finding* | Finding* % [95% CI] |
| 25 | Loss | 46 | 28.1 [21.7, 35.4] |
| 25 | Gain | 39 | 23.8 [17.6, 31.2] |
| 16 | Loss | 22 | 13.4 [8.8, 19.8] |
| 9 | Gain | 21 | 12.8 [8.3, 19.1] |
| 14 | Gain | 18 | 11.0 [6.8, 17.0] |
| 13 | Gain | 14 | 8.5 [4.9, 14.2] |
| 8 | Loss | 13 | 7.9 [4.5, 13.5] |
| 36 | Gain | 10 | 6.1 [3.1, 11.2] |

*Of the 164 dogs with WBC gDNA-specific CNV(s); Findings were not mutually exclusive. Some dogs had multiple concurrent WBC gDNA findings, either within a single chromosome (e.g., a partial gain and a partial loss of chromosome 25) or affecting multiple chromosomes (e.g., gains on both chromosomes 14 and 25)

The study cohort was analyzed to evaluate the normalized age of patients without WBC gDNA-specific CNVs, and their cancer status. Among patients without WBC gDNA-specific CNVs, dogs with a diagnosis of cancer (definitive or presumptive) were significantly older than dogs who submitted samples as part of the clinical cohort for the purpose of screening and had no evidence of cancer (p<0.0001); in addition, patients with WBC gDNA-specific findings (whether or not they had cancer) were significantly older than the cancer-diagnosed dogs without WBC gDNA-specific CNVs (p<0.0001) (FIG. 4). No significant difference was observed in the age distribution of patients with WBC gDNA-specific CNVs as a function of cancer status (p=0.5864).

Embodiments of the methods described herein for large-scale NGS-based genomic profiling of dogs with and without cancer has surprisingly and unexpectedly uncovered evidence of age-related somatic copy number alterations in dogs at the population level.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although embodiments described herein have been disclosed in the context of certain embodiments and examples, those skilled in the art will understand that the present disclosure extends beyond the specifically disclosed embodiments to other alternative embodiments and/or uses of the embodiments described herein and obvious modifications and equivalents thereof. In addition, while several variations of the embodiments have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments described herein. Thus, it is intended that the scope of the present disclosure should not be limited by the particular disclosed embodiments described above.

It should be understood, however, that this detailed description, while indicating some embodiments, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods, and related components. Furthermore, embodiments may include several novel features, no single one of which is solely responsible for its desirable attributes or is believed to be essential to practicing the embodiments herein described.

What is claimed is:

1. A method of detecting a non-cancer somatic mutation in a subject, the method comprising:
obtaining a sample from a subject;
isolating genomic DNA from the sample;
preparing a DNA sequencing library from the genomic DNA (gDNA);
sequencing the gDNA to generate sequencing data; and
detecting at least one copy number aberration in the sequencing data, wherein the at least one copy number aberration is indicative of a non-cancer somatic mutation.

2. The method of claim 1, wherein the subject is a canine subject.

3. The method of claim 1, wherein the sample is a whole blood sample.

4. The method of claim 1, wherein the sequencing data is aligned to a reference genome.

5. The method of claim 1, wherein the gDNA is extracted from white blood cells.

6. The method of claim 5, wherein the white blood cells are present in buffy coat.

7. The method of claim 1, further comprising isolating cell free DNA (cfDNA).

8. The method of claim 7, wherein the cfDNA is extracted from plasma.

9. The method of claim 7, wherein the gDNA is matched with the cfDNA.

10. The method of claim 7, wherein an absence of non-cancer somatic mutations is detected in a cell free DNA sequencing library.

11. A method of detecting a non-cancer somatic mutation in a subject, the method comprising:
isolating white blood cell (WBC) genomic DNA (gDNA) from a buffy coat sample from the subject;
creating a DNA sequencing library from the WBC gDNA;
generating sequencing data by sequencing the DNA sequencing library;
aligning the sequencing data to a reference genome; and
detecting at least one copy number aberration, wherein the at least one copy number aberration is indicative of a non-cancer somatic mutation.

12. The method of claim 11, further comprising:
isolating cell-free DNA (cfDNA) from a plasma sample from the subject;
creating a cfDNA sequencing library from the cfDNA;
generating cell-free sequencing data by sequencing the cfDNA sequencing library;
aligning the cell-free sequencing data to a reference genome; and
detecting an absence of the non-cancer somatic mutations in the cell-free alignment.

13. The method of claim 11, further comprising:
isolating non-buffy coat DNA from a non-buffy coat sample from the subject;
creating a non-buffy coat DNA sequencing library from the non-buffy coat DNA;
generating non-buffy coat sequencing data by sequencing the non-buffy coat DNA sequencing library;
aligning the non-buffy coat sequencing data to a reference genome; and
detecting an absence of the non-cancer somatic mutations in the non-buffy coat alignment.

14. The method of claim 13, wherein one or more non-buffy coat somatic mutations are detected in the non-buffy coat alignment, and wherein the non-buffy coat somatic mutations do not match the non-cancer somatic mutations.

15. The method of claim 11, wherein the subject is a canine subject.

16. The method of claim 11, wherein the buffy coat sample and the plasma sample are matched.

17. A method of measuring an age related somatic alteration in a canine subject, comprising:
measuring a copy number variation (CNV) from white blood cell genomic DNA (WBC gDNA) obtained from a buffy sample from the canine subject; and
detecting an absence of CNV from cell free DNA (cfDNA) obtained from a matched plasma sample from the canine subject.
